(19) United States
(12) Patent Application Publication
(10) Pub. No.: US 2012/0247496 A1
(43) Pub. Date: Oct. 4, 2012

(54) FINGERNAIL AND TOENAIL BASE COAT COMPOSITION AND METHOD OF APPLYING THE SAME

(76) Inventor: Tammy Taylor, Irvine, CA (US)

(21) Appl. No.: 13/074,176

(22) Filed: Mar. 29, 2011

(51) Int. Cl. A61Q 3/02; A61K 8/72

(52) U.S. Cl. 132/200; 424/61

(57) ABSTRACT

Disclosed are compositions and methods for forming a more durable, longer lasting and chip-resistant coating of nail polish upon a fingernail or toenail. The composition comprises a combination of artificial nail builders and photoinitiators, as well as other ingredients, which form a base coat composition. In use, the base coat composition is first applied directly upon the fingernail or toenail and thereafter cured by either UV or LED lighting. Once the base coat is cured upon the fingernail or toenail, conventional nail polish may be applied as desired. The result in cost is exceptionally more robust and chip-resistant than nail polish applied alone.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

Adorning fingernails and toenails with nail polish or paint is well-known and routinely preformed millions of times a day throughout the world. In this regard, countless shades of nail polish are commercially available that are typically designed to be applied directly to the fingernail or toenail surface to thus produce an aesthetically pleasing appearance. To that end, nail polish is typically packaged with a brush applicator that enables the fingernail polish to be applied in a thin layer covering the fingernail or toenail surface. In this regard, the practice of painting fingernails and toenails is exceedingly commonplace and can range from simple self-application to being incorporated as part of a salon-based manicure and pedicure treatment with the nail polish being professionally applied to achieve a desired artistic expression.

As is well-recognized, despite the extensive practice of painting fingernails and toenails, such processes are problematic insofar as the painted coatings are prone to cracking, chipping or otherwise not being able to withstand scratches and other abrasive forces that are encountered by the nails in everyday life. In fact, it is accepted that it is only a matter of time before a painted nail chips or cracks, which in turn produces an aesthetically displeasing nail and otherwise detracts from the remaining painted nails which may otherwise appear perfect.

As a consequence, the fingernail or toenail will be chipped or cracked and requires that the nail polish be removed therefrom, typically via nail polish remover, and again repainted. Alternatively, in the case where nails have been artistically painted by a professional, a single chipped nail can ruin the appearance of all the remaining nails, which in turn requires that all remaining nails essentially need to be redone at a substantial cost.

As such, there is a substantial need in the art for a composition and method that enables a fingernail or toenail surface to be painted in any matter as may be desired whereby the resultant coat of nail polish applied to the nail becomes substantially more durable, robust and chip-resistant to thus substantially prolong the life of the coat of nail polish. There is additionally a need in the art for composition and method that will enable painted nail surfaces to resist cracking or chipping and resist scratches and other abrasive forces to thus enable the wearer to engage in a wide variety of activities without risk of doing damage to the painted nail surface. There is a further need in the art for a method and composition to substantially enhance the strength and durability of a painted nail surface that is time efficient and easy to apply, exceptionally safe, and can either be self-applied or professionally applied in a salon environment, where in either case the resultant painted nail surface will be exceptionally durable and chip-resistant. Finally, there is a need in the art for such a composition and method that can be utilized with virtually all existing nail polish compositions commercially available, especially nitrocellulose-based polishes, and is not in any way limiting to the choices of color or products the wearer may choose to utilize to paint her (or his) fingernail and toenail surfaces.

### BRIEF SUMMARY

The present invention specifically addresses and alleviates the aforementioned deficiencies in the art. In this regard, the present invention is directed to a novel nail base coat composition and method for applying the same that enables conventional nail polish to be applied as desired to the fingernails or toenails, yet results in a substantially more durable, vibrant, longer-lasting and chip-resistant coating of nail polish than can be obtained by conventional nail polish application practices. According to the preferred embodiment, there is first provided a base coat composition comprised of a combination of artificial nail builders and photoinitiators that are preferably formulated as a gel that is applied directly to the fingernail or toenail surface. According to the preferred embodiment, such composition comprises the following ingredients present in the following amounts by a percent weight:

| | | % by |
|---|---|---|
| Ingredients | | weight |
| Bis-HEMA Poly(1,4-Butanediol)-9/ | Artificial Nail Builder | 30-60 |
| IPDI Copolymer | | |
| Hydroxypropyl Methacrylate | Artificial Nail Builder | 15-40 |
| Trimethylolpropane Trimethacrylate | Artificial Nail Builder | 7-13 |
| HEMA | Artificial Nail Builder | 3-7 |
| Methacryloylethyl Phosphate | Artificial Nail Builder | 3-7 |
| Trimethylbenzoyl Diphenyl | photoinitiator | 3-7 |
| Phosphine Oxide | | |
| Benzoyl Isopropanol | photoinitiator | 1-5 |
| Oligomeric alpha-hydroxy ketone | photoinitiator | 1-5 |
| Cellulose Acetate Butyrate | Artificial Nail Builder | 0.5-1.5 |
| substituted benzophenone | photoinitiator | 0.5-1.5 |
| Bis (t-Butyl Benzoxazolyl) Thiophene | fluorescent brightener | PPM |
| | (Optional) | |

As will be understood, the combination of all ingredients will total 100%. In a more highly refined embodiment, the composition comprises the formulation below indicating the relative percent by weight of each component, wherein the sum total of each component will equal 100% by weight:

| | | % by |
|---|---|---|
| Ingredients | | weight |
| Bis-HEMA Poly(1,4-Butanediol)-9/ | Artificial Nail Builder | 40-50 |
| IPDI Copolymer | | |
| Hydroxypropyl Methacrylate | Artificial Nail Builder | 18-30 |
| Trimethylolpropane Trimethacrylate | Artificial Nail Builder | 7-10 |
| HEMA | Artificial Nail Builder | 4-6 |
| Methacryloylethyl Phosphate | Artificial Nail Builder | 4-6 |
| Trimethylbenzoyl Diphenyl | photoinitiator | 3-6 |
| Phosphine Oxide | | |
| Benzoyl Isopropanol | photoinitiator | 2-4 |
| Oligomeric alpha-hydroxy ketone | photoinitiator | 2-4 |
| Cellulose Acetate Butyrate | Artificial Nail Builder | 0.5-1.5 |
| substituted benzophenone | photoinitiator | 0.5-1.5 |
| Bis (t-Butyl Benzoxazolyl) Thiophene | fluorescent brightener | PPM |
| | (Optional) | |

In a most highly preferred embodiment, the composition comprises the following listed ingredients as provided in the relative percentage by weight, wherein the combination of all ingredients totals 100%.

| Ingredients | % by weight |
|---|---|
| Bis-HEMA Poly(1,4-Butanediol)-9/IPDI Copolymer | 46% |
| Hydroxypropyl Methacrylate | 24% |
| Trimethylolpropane Trimethacrylate | 8% |
| HEMA | 6% |
| Methacryloylethyl Phosphate | 4.5% |
| Trimethylbenzoyl Diphenyl Phosphine Oxide | 4% |
| Benzoyl Isopropanol | 3.5% |
| Oligomeric alpha-hydroxy ketone | 2.5% |
| Cellulose Acetate Butyrate | 1.0% |
| substituted benzophenone | 0.5% |

Although not essential, it is contemplated that other components may also be added to the base coat composition as may be desired to produce certain qualities and characteristics. In this regard, it is contemplated that fragrances, coloring agents and thixotropic agents may be added to respectively produce a desired color, fragrance or composition thickness. Other well-known additives, such as preservatives, antioxidants and the like may also be included as desired in quantities that can be readily determined with those skilled in the art.

The composition will preferably be generally gel-like in nature and may be applied by any of variety techniques known in the art. Most typically, the base coat composition may be painted or brushed directly on the fingernail or toenail surface that will ultimately receive the nail polish per conventional brushing application processes. In this regard, the base coat need only be applied in sufficient thickness to cover the surface of the nail to be painted.

Once the base coat has been applied to the nail, the base coating is then subjected to a curing treatment. With respect to the later, such curing treatment comprises of subjecting the base coat composition as applied to the nail to a UV or LED light source for a time sufficient to activate the photoinitiators present within the composition to polymerize the artificial nail builder components of the base coat composition. In this regard, it is contemplated that the artificial nail builder components will essentially comprise polymerizeable compounds to thus form a polymeric coat that will ultimately form the surface upon which the nail polish is applied. With respect to the curing process, the same may be accomplished by any of a variety of conventional UV and/or LED lighting sources and for curing via application of UV exposure, it is contemplated that the curing step may be accomplished from anywhere from 1 to 3 minutes exposure to UV lighting, with approximately 2 minutes being most preferred. With respect to curing LED lighting, the base coat composition will preferably be subjected to such lighting from 20 to 60 seconds, with 40 seconds being preferred.

After the base coat composition has been allowed to cure upon the fingernails and/or toenails to be painted, conventional nail polish may then be applied per conventional prior art practices. In this regard, one or more nail polish compositions need only be brushed upon the fingernail or toenail surface and allowed to dry as is common practice. Advantageously, the base coat composition is completely compatible with all types of commercially available nail polish, especially the most common nitrocellulose-based polishes, and it is expressly contemplated that the compositions and methods of the present invention can be readily deployed in existing nail salons, or alternatively, may be self-applied with a minimal time and effort required.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a schematic diagram describing the steps and sequence for applying the fingernail base cost composition of the present invention and for forming an exceptionally robust and chip-resistant coating of fingernail polish.

### DETAILED DESCRIPTION

The above description is given by way of example, and not limitation. Given the above disclosure, one skilled in the art could devise variations that are within the scope and spirit of the invention disclosed herein, including various ways of forming a base coat upon a fingernail or toenail surface for increasing the durability of a coat of nail polish applied thereto. Further, the various features of the embodiments disclosed herein can be used alone, or in varying combinations with each other and are not intended to be limited to the specific combination described herein. Thus, the scope of the claims is not to be limited by the illustrated embodiments.

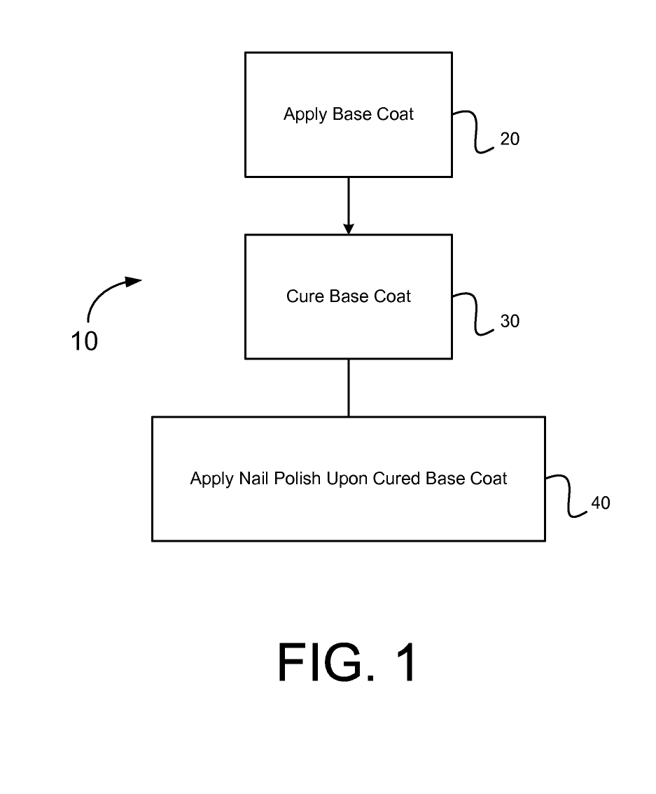

Referring now to FIG. 1, there is shown a process 10 for forming a coating of nail polish upon the surface of a nail (i.e., fingernail or toenail) wherein the resultant coating formed upon the nail is exceptionally more durable, chip-resistant and robust compared to prior art practices for painting and applying nail polish to nails.

The process 10 comprises the initial step 20 of applying a base coat composition to the nail surface to which the nail polish or paint will ultimately be applied. The composition as applied comprises a combination of artificial nail builders, which typically comprise polymeric precursor agents, and photoinitiators that, discussed more fully below, are operative to form a base coat on top of the nail surface to which the fingernail polish or paint is ultimately applied.

With respect to the ingredients of such compositions, the same are set forth below wherein each ingredient the specified in its relative amount by percent weight:

| | | % by |
|---|---|---|
| Ingredients | | weight |
| Bis-HEMA Poly(1,4-Butanediol)-9/ | Artificial Nail Builder | 30-60 |
| IPDI Copolymer | | |
| Hydroxypropyl Methacrylate | Artificial Nail Builder | 15-40 |
| Trimethylolpropane Trimethacrylate | Artificial Nail Builder | 7-13 |
| HEMA | Artificial Nail Builder | 3-7 |
| Methacryloylethyl Phosphate | Artificial Nail Builder | 3-7 |
| Trimethylbenzoyl Diphenyl | photoinitiator | 3-7 |
| Phosphine Oxide | | |
| Benzoyl Isopropanol | photoinitiator | 1-5 |
| Oligomeric alpha-hydroxy ketone | photoinitiator | 1-5 |
| Cellulose Acetate Butyrate | Artificial Nail Builder | 0.5-1.5 |
| substituted benzophenone | photoinitiator | 0.5-1.5 |
| Bis (t-Butyl Benzoxazolyl) Thiophene | fluorescent brightener | PPM |
| | (Optional) | |

In a more preferred embodiment, the composition comprises the following ingredients whereas in the relative amount by percent weight:

| | | % by |
|---|---|---|
| Ingredients | | weight |
| Bis-HEMA Poly(1,4-Butanediol)-9/ | Artificial Nail Builder | 40-50 |
| IPDI Copolymer | | |
| Hydroxypropyl Methacrylate | Artificial Nail Builder | 18-30 |
| Trimethylolpropane Trimethacrylate | Artificial Nail Builder | 7-10 |
| HEMA | Artificial Nail Builder | 4-6 |
| Methacryloylethyl Phosphate | Artificial Nail Builder | 4-6 |
| Trimethylbenzoyl Diphenyl | photoinitiator | 3-6 |
| Phosphine Oxide | | |
| Benzoyl Isopropanol | photoinitiator | 2-4 |
| Oligomeric alpha-hydroxy ketone | photoinitiator | 2-4 |
| Cellulose Acetate Butyrate | Artificial Nail Builder | 0.5-1.5 |
| substituted benzophenone | photoinitiator | 0.5-1.5 |
| Bis (t-Butyl Benzoxazolyl) Thiophene | fluorescent brightener | PPM |
| | (Optional) | |

In a most highly preferred embodiment, the composition comprises the formulation below wherein each ingredient is present in amount by percent weight as indicated:

| Ingredients | % by weight |
|---|---|
| Bis-HEMA Poly(1,4-Butanediol)-9/IPDI Copolymer | 46% |
| Hydroxypropyl Methacrylate | 24% |
| Trimethylolpropane Trimethacrylate | 8% |
| HEMA | 6% |
| Methacryloylethyl Phosphate | 4.5% |
| Trimethylbenzoyl Diphenyl Phosphine Oxide | 4% |
| Benzoyl Isopropanol | 3.5% |
| Oligomeric alpha-hydroxy ketone | 2.5% |
| Cellulose Acetate Butyrate | 1.0% |
| substituted benzophenone | 0.5% |
| Violet 2 (CI 60725) (Optional) | PPM |

In all instances, it is understood that the ingredients will total 100% by weight. Moreover, it is further understood that the composition may comprise one or more additives well-known to the those skilled in the art known to provide a desired characteristic or property of the base coat composition. For example, it is contemplated that additives such as fragrances, colorants, thixotropic agents, preservatives, antioxidants and any of a variety of combinations thereof may be added to the composition to thus enhance its properties. As should be understood, such additives are not essential and neither contribute to nor detract from the ability of the specified ingredient set forth above to ultimately produce the desired base coat composition upon which the nail paint or polish is to be applied.

With respect to the application of such composition, the same may be accomplished by any of a variety of means well-known to the art. Typically, it is contemplated that such composition need only be brushed upon the nail surface using conventional fingernail/toenail brushes well-known in the art and commercially available in virtually every retail establishment where cosmetics and nail care products are sold. With respect to the application of the base coat composition, the same need only be applied in an amount sufficient to cover the nail surface as such nail coatings are typically applied. In this regard, the base coat composition need only be applied as a single layer and need not be applied with any excessive thickness, multiple applications or the like. Essentially, provided that the layer is brushed upon the nail surface the amount sufficient to cover the nail surface, the base coat composition should be deemed to have been adequately applied.

Following the application of the base coat 20, the next step in the process 10 is a curing step 30. To perform such step, the nails with base coat compositions applied thereon are subjected to a light source sufficient to activate the photoinitiators to cause polymerization of the specified artificial nail builder component of the composition. To that end, it is contemplated that the curing step 30 may be accomplished by a variety of means, but particularly may involve subjecting the base coat composition, as applied to the nail surface, to either UV or LED lighting. With respect to such lighting sources, the same are commercially available and well-known to selectively direct a beam of either UV or LED lighting onto a fingernail or toenail surface. With respect to UV lighting, it is contemplated that the base coat composition should be subjected from 1 to 3 minutes, with 2 minutes being most preferred. With respect to LED lighting, it is contemplated that the base coat composition need be exposed thereto for a duration of 20 to 60 seconds, with 40 seconds being most preferred. In this regard, such exposure for either application of UV or LED lighting is believed to be sufficient to complete polymerization of the base coat composition upon the nail surface.

Following the curing step 30, the desired nail polish or paint may then be applied upon the cured base coat as may be desired in step 40. To that end, step 40 will essentially be painting the fingernail or toenail surface as is routinely practiced in the prior art, and is not restricted in anyway to any type of nail polish or paint composition. In this regard, the base coat compositions of the present invention, when cured upon the nail surface, are compatible with all known types of commercially available nail polishes, especially nitrocellulose-based polishes, whether clear or with colorants, and the wearer or nail polish applier may utilize any commercially available product as may be desired, including combinations thereof, to adorn the nail surface. In this regard, it is contemplated that the compositions and methods of the present invention will essentially enable fingernails and toenails to be painted in any manner desired via conventional practices, and by virtue of the application of the base coat, will result in a nail polish coating that is formed in such a manner that is substantially more durable, robust and chip-resistant coating that the wearer can enjoy for substantially longer use and enjoyment, and can further provide a substantially reduced risk of cracking or peeling compared to existing nail polishes applied directly to the nail surface.

Advantageously, despite being exceptionally more durable and chip-resistant, the fingernail polish coatings of the present invention may also be removed at any time desired by the wearer using conventional nail polish removers and the like. In this regard, the fingernail polish or paint applied to the cured base coat in the aforementioned manner does not increase the difficulty by which the nail polish can be removed and despite being more durable and chip-resistant, the fingernail polish or paint is no more difficult to remove than conventional fingernail polish and paints applied directly to fingernail and toenail surfaces. As such, as will be appreciated by those skilled in the art, there are no drawbacks or complications with nail polish or paint removal when utilizing the composition of the methods of the present invention to form the desirable painted nail finishes discussed herein.

What is claimed is:

1. A method for forming a chip-resistant coating of nail polish upon a fingernail or toenail surface comprising:
a) applying a base coat upon said fingernail or toenail surface, said base coat composition comprising:

| | Percent range |
|---|---|
| Ingredients | by weight |
| Bis-HEMA Poly(1,4-Butanediol)-9/IPDI Copolymer | 30-60 |
| Hydroxypropyl Methacrylate | 15-40 |
| Trimethylolpropane Trimethacrylate | 7-13 |
| HEMA | 3-7 |
| Methacryloylethyl Phosphate | 3-7 |
| Trimethylbenzoyl Diphenyl Phosphine Oxide | 3-7 |
| Benzoyl Isopropanol | 1-5 |
| Oligomeric alpha-hydroxy ketone | 1-5 |
| Cellulose Acetate Butyrate | 0.5-1.5 |

b) curing said base coat applied in step a); and
c) applying a coating of nail polish upon said base coat cured in step b).

2. The method of claim 1 wherein in step a), said composition comprises:

| | Percent range |
|---|---|
| Ingredients | by weight |
| Bis-HEMA Poly(1,4-Butanediol)-9/IPDI Copolymer | 40-50 |
| Hydroxypropyl Methacrylate | 18-30 |
| Trimethylolpropane Trimethacrylate | 7-10 |
| HEMA | 4-6 |
| Methacryloylethyl Phosphate | 4-6 |
| Trimethylbenzoyl Diphenyl Phosphine Oxide | 3-6 |
| Benzoyl Isopropanol | 2-4 |
| Oligomeric alpha-hydroxy ketone | 2-4 |
| Cellulose Acetate Butyrate | 0.5-1.5 |

3. The method of claim 1 wherein in step a), said base coat composition comprises:

| Ingredients | % by weight |
|---|---|
| Bis-HEMA Poly(1,4-Butanediol)-9/IPDI Copolymer | 46% |
| Hydroxypropyl Methacrylate | 24% |
| Trimethylolpropane Trimethacrylate | 8% |
| HEMA | 6% |
| Methacryloylethyl Phosphate | 4.5% |
| Trimethylbenzoyl Diphenyl Phosphine Oxide | 4% |
| Benzoyl Isopropanol | 3.5% |
| Oligomeric alpha-hydroxy ketone | 2.5% |
| Cellulose Acetate Butyrate | 1.0% |
| substituted benzophenone | 0.5% |
| Violet 2 (CI 60725) | PPM |

4. The method of claim 1 wherein in step b), said step of curing is accomplished by exposing said base composition as applied to said fingernail or toenail to UV light.

5. The method of claim 1 wherein in step b), said step of curing is accomplished by exposing said base composition as applied to said fingernail or toenail to LED light.

6. The method of claim 4 wherein said UV light is emitted upon said base coat composition for between 1 to 3 minutes.

7. The method of claim 6 wherein said UV light is emitted upon said base coat composition for approximately 2 minutes.

8. The method of claim 5 wherein said base coat is exposed to said LED lighting for between 20 seconds to 60 seconds.

9. The method of claim 8 wherein said base coat is exposed to said LED lighting for approximately 40 seconds.

10. A composition for forming a base coat upon a fingernail or toenail surface comprising:

| | Percent range |
|---|---|
| Ingredients | by weight |
| Bis-HEMA Poly(1,4-Butanediol)-9/IPDI Copolymer | 30-60 |
| Hydroxypropyl Methacrylate | 15-40 |
| Trimethylolpropane Trimethacrylate | 7-13 |
| HEMA | 3-7 |
| Methacryloylethyl Phosphate | 3-7 |
| Trimethylbenzoyl Diphenyl Phosphine Oxide | 3-7 |
| Benzoyl Isopropanol | 1-5 |
| Oligomeric alpha-hydroxy ketone | 1-5 |
| Cellulose Acetate Butyrate | 0.5-1.5 |

11. The base coat composition of claim 10 wherein said composition comprises:

| | Percent range |
|---|---|
| Ingredients | by weight |
| Bis-HEMA Poly(1,4-Butanediol)-9/IPDI Copolymer | 40-50 |
| Hydroxypropyl Methacrylate | 18-30 |
| Trimethylolpropane Trimethacrylate | 7-10 |
| HEMA | 4-6 |
| Methacryloylethyl Phosphate | 4-6 |
| Trimethylbenzoyl Diphenyl Phosphine Oxide | 3-6 |
| Benzoyl Isopropanol | 2-4 |
| Oligomeric alpha-hydroxy ketone | 2-4 |
| Cellulose Acetate Butyrate | 0.5-1.5 |

12. The composition of claim 10 wherein said composition comprises:

| Ingredients | % by weight |
|---|---|
| Bis-HEMA Poly(1,4-Butanediol)-9/IPDI Copolymer | 46% |
| Hydroxypropyl Methacrylate | 24% |
| Trimethylolpropane Trimethacrylate | 8% |
| HEMA | 6% |
| Methacryloylethyl Phosphate | 4.5% |
| Trimethylbenzoyl Diphenyl Phosphine Oxide | 4% |
| Benzoyl Isopropanol | 3.5% |
| Oligomeric alpha-hydroxy ketone | 2.5% |
| Cellulose Acetate Butyrate | 1.0% |
| substituted benzophenone | 0.5% |
| Violet 2 (CI 60725) | PPM |
